(19) United States
(12) Patent Application Publication
(10) Pub. No.: US 2012/0123219 A1
(43) Pub. Date: May 17, 2012

(54) METHOD AND SYSTEM FOR CONTROLLING MEDICAL MONITORING EQUIPMENT

(76) Inventors: Emil Markov Georgiev, Hartland, WI (US); Timothy Patrick McCormick, Fitchburg, WI (US); Joel Patrick Wenzl, Sun Prairie, WI (US); Maureen Marie Domanico, Middleton, WI (US); Fabrizio Redaelli, Milan (IT); Jerome Boda Korten, New York, NY (US)

(21) Appl. No.: 12/946,023

(22) Filed: Nov. 15, 2010

(51) Int. Cl. A61B 5/00; G06F 3/041; H04Q 5/22; G06F 3/00; G06F 3/048

(52) U.S. Cl. 600/300; 715/700; 715/867; 715/764; 340/10.1; 345/173

(57) ABSTRACT

A device for monitoring physiological parameters of a medical patient includes a pneumatic system configured to be coupled to a patient to provide a regulated gas thereto, a computer coupled to the pneumatic system and configured to regulate gas to the patient via the pneumatic system, and a touchscreen monitor coupled to the computer. The touchscreen monitor includes a first graphical user interface (GUI) having a first display, and a second GUI having a second display different from the first display and configured having interaction fields to enable parameters to be input therewith. The device includes a first trigger configured to switch at least from the first GUI to the second GUI.

### BACKGROUND OF THE INVENTION

Embodiments of the invention relate generally to equipment for monitoring physiological parameters of a medical patient and, more particularly, to an apparatus and method of accessing and controlling a user interface for monitoring and treatment equipment in a medical environment.

Equipment for monitoring physiological parameters of a medical patient and patient treatment include ventilators, anesthesia machines, and vital signs monitoring equipment that are often located in a hospital or medical environment. The environment may include an intensive care unit (ICU) or a pediatric ward, as examples. A patient is monitored in the environment for extended periods of time while medical personnel pass in and out of the environment during a normal course of business.

Patients that have respiratory difficulties often are placed on a ventilator. These respiratory difficulties may be pathological in nature or may be due to the fact that the patient is too weak or sedated to independently perform respiration functions. Often, the patient may be spontaneously attempting to breathe but is not able to complete a full respiratory cycle. In these cases, mechanically assisted ventilation is provided. In some mechanically assisted ventilation platforms, a combination of pressure and/or flow sensors detect a patient's attempt to breath. Detection of a breath attempt triggers mechanical delivery of the breath. The breath is provided by the delivery of medical gases under a pressure that is sufficient to overcome system resistance and the patient's airway resistance to fill the lungs in an inspiratory phase. When the pressure of the medical gas is reduced, the natural elasticity of the patient's chest wall forces the delivered breath out of the patient in an expiratory phase.

Thus, in patient monitoring equipment, medical gases may be supplied to a patient that include air, oxygen, helium, nitric oxide, anesthetic agent, drug aerosol, or any other gas breathed by the patient. Oxygen is typically referred to as the drive gas for the ventilator system and other medical gases are typically referred to as supplemental gases to the air. There are currently a wide variety of systems available to provide ventilator support to a patient, to provide anesthesia delivery to a patient, and to monitor vital signs of a patient.

Often, such equipment includes a graphical user interface (GUI) that includes display of both monitoring information (waveforms, actual parameter levels, etc. . . . ) as well as navigation, command, and settings levels, as examples. However, there may be a different context of use for such equipment. For instance, there may be a setup mode to establish initial parameter settings for general system operation. There may also be an adjustment mode where parameters may be set or established for, for instance, a specific patient or a changed monitoring condition. There may also be a monitoring mode where a user need only see operating waveforms, setpoints, actual operating levels, and the like. Moreover, there may be conditions where it is desirable to be able to view this information from a distance (e.g., in an ICU isolation ward), while in other conditions it may be desirable to view up close and in small text, as an example.

Thus, there may be numerous display modes or settings for monitoring and treatment equipment in a medical environment, and the desired display settings are often specific to the type of personnel that are in proximity to the patient or depending on the environment in which the patient is placed. For instance, a nurse may desire to regularly view and have the ability to change equipment setpoints during steady-state or stationary operation, while a doctor may desire realtime access to waveforms or full medical ventilator functionality in an emergency situation. Or, a patient may be positioned in a separate environment (e.g., behind a glass window) from personnel who regularly need to simply view patient vital signs or other information to get a quick snapshot of a patient condition. Additionally, as another example, some users may desire a customized setting to provide both monitoring parameters and an ability to set parameters in a combination that is specific and unique to a user.

As such, varying amounts of information are available for viewing, and different users of monitoring and treatment equipment often desire access to different types of information and settings. However, it is often not feasible to provide all such information in a single GUI setting because of the different types of personnel who may interact with the patient. Thus, GUI settings may be set to include excess information for any specific user in order that all such information is available for any potential user. Accordingly, the available information may be presented in a fashion that is therefore not optimized for any user, or displayed information may be optimized for a single user which then is non-optimal for other users. Or, information may be available for other users, but obtained only after a cumbersome manual switch that may require product expertise and specific knowledge in order to configure the display in the desired fashion.

Therefore, it would be desirable to design an apparatus and method of data visualization and control of medical monitoring and treatment equipment that overcomes the aforementioned drawbacks.

### BRIEF DESCRIPTION

The invention is a directed method and apparatus for interfacing with monitoring equipment.

According to one aspect of the invention, a device for monitoring physiological parameters of a medical patient includes a pneumatic system configured to be coupled to a patient to provide a regulated gas thereto, a computer coupled to the pneumatic system and configured to regulate gas to the patient via the pneumatic system, and a touchscreen monitor coupled to the computer. The touchscreen monitor includes a first graphical user interface (GUI) having a first display, and a second GUI having a second display different from the first display and configured having interaction fields to enable parameters to be input therewith. The device includes a first trigger configured to switch at least from the first GUI to the second GUI.

According to another aspect of the invention, a method of monitoring physiological parameters of a patient includes displaying a first data display in a first graphical user interface (GUI) of a touchscreen monitor, and triggering the touchscreen monitor to display a second GUI that includes a second data display that is different from the first data display, and includes one or more interaction fields in the second GUI, wherein the one or more interaction fields are configured to set parameters of a device for monitoring the physiological parameters of the medical patient.

According to yet another aspect of the invention, a non-transitory computer readable storage medium having stored thereon a computer program representing a set of instructions that when executed by a computer causes the computer to display a first graphical user interface (GUI) of a touchscreen display having a first data display, display a second GUI of the touchscreen display having a second data display that is different from the first data display, and having one or more interaction fields for parameter input, and receive an input from a first trigger that is configured to switch at least between the first GUI and the second GUI.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 illustrates a pictorial view of a ventilator incorporating embodiments of the invention.

FIG. 2 illustrates a block diagram of a ventilator incorporating embodiments of the invention.

FIGS. 3 and 4 are examples of a graphical user interface (GUI).

FIG. 5 is a control algorithm that may be implemented by a controller to control hospital monitoring equipment according to the invention.

### DETAILED DESCRIPTION

The operating environment of the invention is described with respect to a bedside ventilator unit. However, it will be appreciated by those skilled in the art that the invention is equally applicable for use with any hospital monitoring equipment such as anesthesia machines and vital signs monitoring equipment that may be controlled by a computer having interaction via a touchscreen monitor or a graphical user interface (GUI).

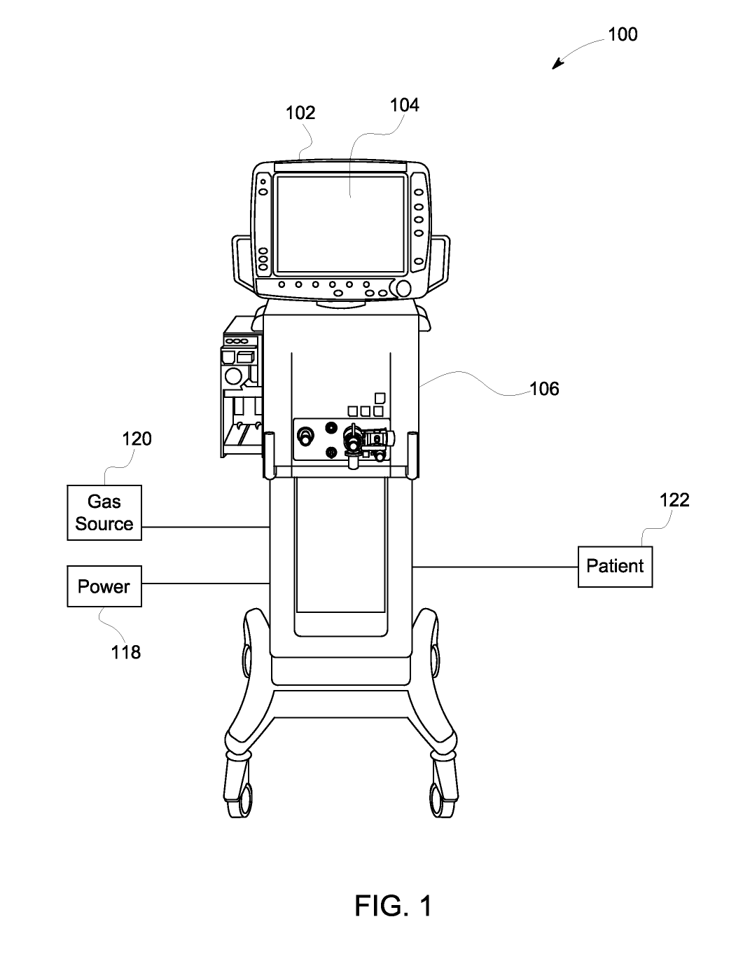

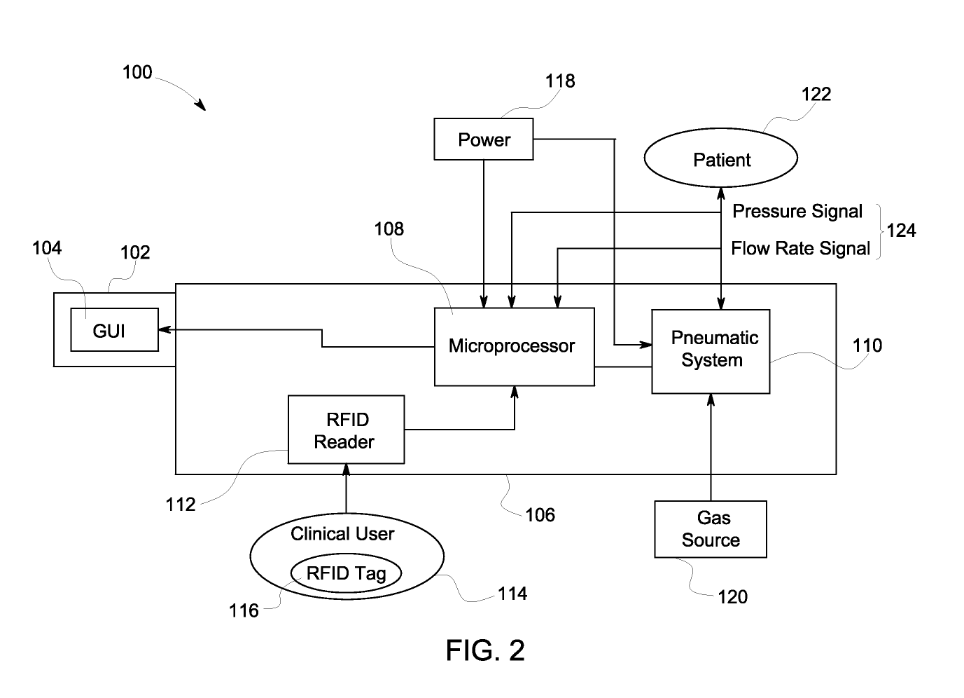

Referring to FIGS. 1 and 2, respectively a pictorial view and a block diagram of a hospital monitoring system incorporating embodiments of the invention is illustrated. And, although the system illustrated is referred to as a ventilator, it is contemplated that the system illustrated may be an anesthesia machine or a vital signs monitoring machine incorporating embodiments of the invention. Ventilator 100 includes a display 102 that includes a screen 104. Screen 104 includes a touch-sensitive screen, or touchscreen, that may be used to input parameters for control of ventilator 100 via a graphical user interface (GUI) which may be used to display set parameters, operating conditions, patient information, and the like. Ventilator 100 includes a ventilator housing 106 that may include a microprocessor or controller 108 and a pneumatic system 110 coupled thereto. Ventilator housing 106 includes, according to one embodiment, a radio-frequency identification (RFID) unit 112 positioned therein that may be set up to activate when an RFID tag is sensed within, for instance, 1.5 meters of ventilator 100. According to other embodiments of the invention, unit 112 may include instead a proximity sensor such as an infrared sensor, an optical sensor, a laser, a bluetooth device, and the like. And, it is to be recognized that unit 112 is not to be limited to the aforementioned devices, but may be any device that is configured to interact with a computing device or a monitoring device. Thus, unit 112 may be configured to receive information from a clinical user 114 via an RFID tag 116 (in the case of an RFID unit 112) or via a another signal that corresponds to the use of a proximity sensor, as is understood in the art. FIGS. 1 and 2 also illustrate a power source 118 configured to power unit 100, and a gas source 120 coupled to pneumatic system 110, being regulated thereby in order to provide a regulated gas flow to a patient 122.

In operation, control parameters are input to ventilator 100 via screen 104, and microprocessor or controller 108 controls operation of ventilator 100. Gas thereby flows from gas source 120 to patient 122 in a regulated fashion via pneumatic system 110 as understood in the art, based on operating parameters set in screen 104 and based on pressure signals, flow rate signals, and the like 124 as illustrated.

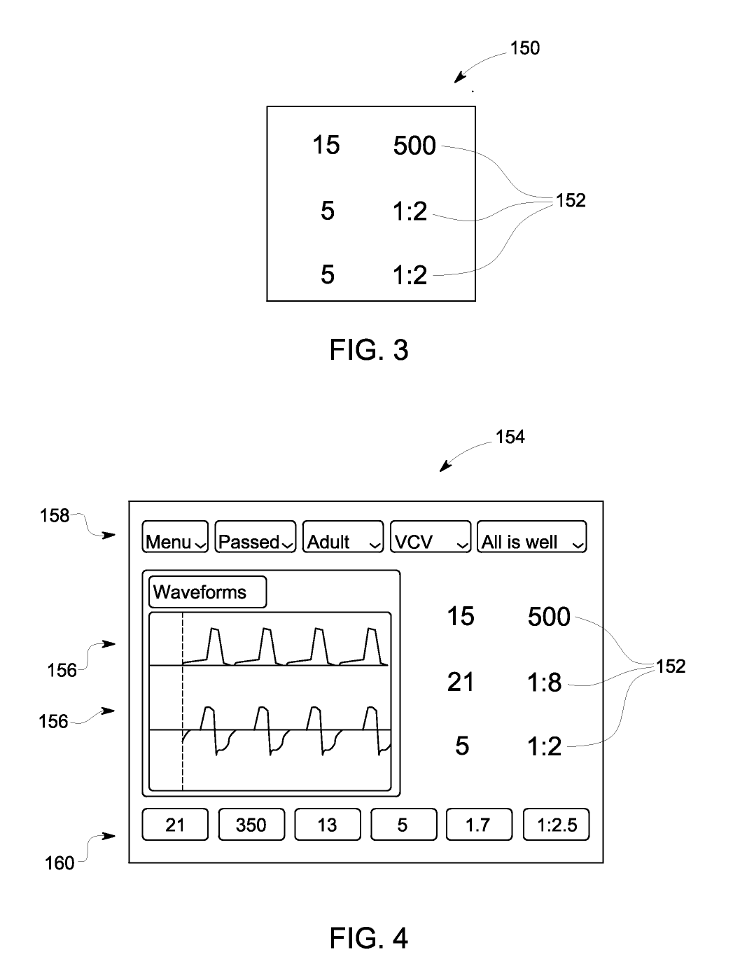

Different operators of ventilator 100 may desire different GUIs to be displayed on screen 104. For instance, referring to FIG. 3, a GUI 150 is illustrated having only monitoring data and command structures 152 illustrated. Thus, GUI 150 may be a desirable display in, for instance, an intensive care unit (ICU) room or in an isolation ward to allow simple and clear visualization of monitoring data at a distance. Such a viewing option may be desirable when a monitoring unit is positioned in a room and where personnel may view GUI 150 from outside the room and through a window, avoiding the necessity to enter the room in order to see monitoring data of ventilator 100.

However, another operator may desire a different GUI than that displayed as GUI 150 in FIG. 3. Thus, referring to FIG. 4, a GUI 154 may include not only monitoring data and command structures 152 (as illustrated in FIG. 3), but also waveforms 156, a control panel 158, and input fields 160. Thus, GUI 154 may be configured for a different user that desires additional data for patient monitoring (e.g., waveforms 156), as well as an ability to modify settings of ventilator 100 via input fields 160, and an ability to drive to other menu options via control panel 158.

It is contemplated that there may be many desirable GUI displays, and not only those illustrated in FIGS. 3 and 4. For instance, according to one embodiment, a superuser may be designated who may have an overriding control having yet additional authority to affect control of ventilator 100. In one example, such a superuser may be a maintenance technician who is performing calibration or other maintenance on ventilator 100, and may thus desire access to operating information that is not normally needed by medical personnel during a monitoring operation. In another example, such a superuser may be a supervising official (doctor or head nurse) who may desire to limit access to different personnel or even exclude certain personnel from access (e.g., a janitor who may inadvertently bump the machine and alter a setting). Thus, there may be numerous GUIs that may be desirable to be set for ventilator 100, based on which user may desire access thereto and based on desired usage for different users. As such, operation of display 102 may be controlled according to embodiments of the invention as illustrated in a GUI control algorithm 200.

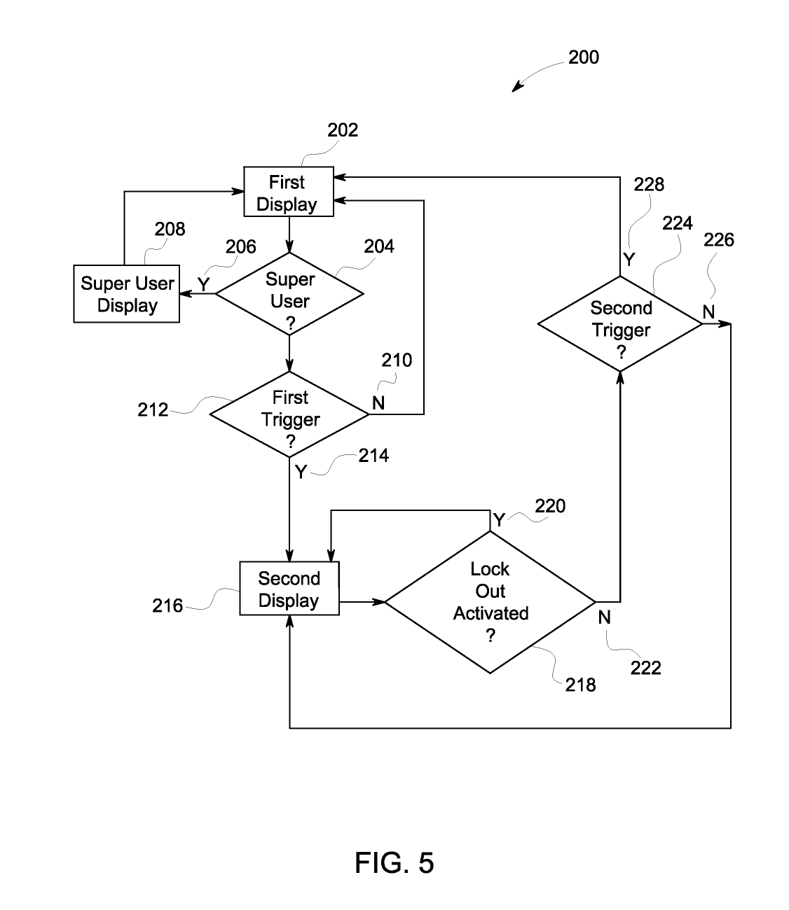

FIG. 5 illustrates a control algorithm that may be implemented by microprocessor or controller 108 of ventilator 100, according to embodiments of the invention. Referring now to FIG. 5, a first display 202 may include a GUI display that corresponds to, for instance, GUI 150 illustrated in FIG. 3. In this mode, a first display of GUI 150 may be set for relatively longer-term viewing during periods when hospital personnel desire to monitor data of a patient without a desire to input a change in parameters.

Change from first display may be affected by use of an RFID tag (or a proximity sensor), as described. For purposes of brevity, an RFID tag will be described henceforth as that used to trigger a new GUI display (although an RFID tag is referenced henceforth, it is to be understood that such change may be via any sensor, such as a proximity sensor, as discussed). When an RFID tag is sensed in proximity to ventilator 100, GUI control algorithm 200 determines whether the sensed RFID tag is a superuser 204. If so 206, then a superuser GUI is displayed 208, after which control returns to first display 202. However, if not 210, then control algorithm 200 determines whether the sensed RFID tag is a first trigger 212. If not, then control returns to first display 202. In other words, in a first display 202 mode, according to an embodiment of the invention, GUI control algorithm 200 displays first display 202 that may correspond to, for instance, GUI 150. When an RFID tag is sensed in proximity to ventilator 100, then GUI control algorithm 200 may bring up superuser display 208, or may determine if the sensed RFID tag is first trigger 212. If so 214, then a second display 216 may include a GUI such as GUI 154 illustrated in FIG. 4. And, it is contemplated that second display 216 may be set to correspond to a specific RFID tag. That is, a user A may have a desired GUI for second display 216 that is different from a user B. For instance, one user may be a nurse who desires vital signs, status, and limited ability to set parameters, while another user may be a pulmonologist who desires access to waveforms and more details. Thus, second display 216 may be tailored or customized to each type of user or even to an individual. Thus, when an RFID tag is sensed 214, a GUI may be displayed that corresponds to a specific user and their specific GUI or to a user type. As such, there may be multiple second displays 216 that may be activated by first trigger 212. Second display 216 may also be activated to a default GUI by tapping on screen 104 (for instance, for a user that does not have an RFID tag), according to an embodiment of the invention.

Furthermore, it is contemplated that multiple users/RFID tags may be sensed in proximity to ventilator 100 simultaneously. Thus, to avoid confusion and inadvertently triggering an undesired GUI, users/RFID tags may be assigned a priority to ensure a quick transition to a desired GUI. That is, if users A and B are sensed simultaneously, it is contemplated that, for instance, user A may be assigned a higher command priority than user B. As such, sensing first trigger 212 may also include sensing a command priority assigned to each RFID tag, according to embodiments of the invention. In such fashion, control may seamlessly pass to a desired GUI according to embodiments of the invention.

In addition, it is contemplated that control between priority RFID tags may be yet further controlled by implementing an overriding priority assessment. For example, if a lower command priority user B is first sensed in proximity to ventilator 100 and user B's GUI is displayed, but a short while later (e.g., a few seconds or minutes later) a higher command priority user A is sensed in proximity to ventilator 100, then it is contemplated that user A's GUI may be displayed. Such functionality may be in, for instance, an emergency situation when multiple users may converge in proximity to ventilator 100, thus allowing a desired authorized user to gain immediate access to full medical ventilator functionality.

However, a lower priority user may be in the process of working with a patient on ventilator 100. Thus, it is contemplated that a user may lock out 218 other users (having a higher command priority) from inadvertently switching to a different GUI. Thus, if a lower priority user B is working on user B's GUI, then user B may lock out a higher priority user A from overriding to user A's GUI in order to avoid overriding to user A's GUI and potentially causing injury to a patient. Thus, if lock out 218 is activated 220, then second display 216 will maintain the current GUI so that user B may continue working in an uninterrupted fashion. Lock out may be implemented by user B even if a higher priority user or even a superuser is sensed in proximity to ventilator 100. However, it is also contemplated that user B may, in this example, actively switch to user A's GUI by having a selectable field that can switch to another user GUI. Further, lock-out 218 may be activated in any number of fashions, to include an active trigger on a user's GUI, or by the simple act of interacting with the user's GUI.

If no lock-out is activated 222, then control continues to a second trigger 224. However, if no second trigger is activated 226, then control returns to second display 216 and its corresponding GUI. However, if a second trigger is activated 228, then control returns to first display 202. Second trigger 224 may be activated after a fixed time of inactivity, after no RFID tag is sensed, or a combination thereof. Second trigger 224 may also be actively activated by a user of second display 216. That is, when a user has finished using second display 216, then a field on second display 216 may enable a user to switch to first display 202 by selecting a field or button within the GUI of second display 216.

In addition, it is contemplated that RFID tags may have a corresponding alarm type associated therewith. For instance, if a user B is working on a patient and a user A RFID tag is sensed, then a brief chirp or beep may be activated to alert the users that user A has been sensed, while still not overriding to user A GUI. The brief chirp or beep may be coded to each user or type of user, according to the invention.

Thus, multiple GUIs for a touchscreen ventilator may be controlled and navigated according to the invention. The multiple GUIs may include simple data display, limited navigation and parameter setting, or detailed display areas, which depend on what a desired use is. Activation parameters may be set based on the type of user detected in the presence of the equipment, and types of users may have established priorities. Users may have the ability to lock out other users, even if the users being locked out have a higher command priority than the current user. Triggering between GUIs may be via RFID tags, proximity sensors, or by tapping the touchscreen. Types of triggering may also include a screen saver mechanism where a monitoring GUI mode is activated based on a pre-set time lapse of user inactivity—and an interaction mode may be thereby re-activated by again tapping the touchscreen. Monitoring mode may also be activated when no RFID tags are sensed in the proximity of ventilator 100. Furthermore, RFID triggering may be tailored to specific RFID tags, or classes of tags (e.g., multiple ‘user B’ types). Thus, a user may use a GUI that is in a setup mode having a full interaction GUI (e.g., for setting monitoring parameters) and then switched to a monitoring mode having a display GUI.

A technical contribution for the disclosed method and apparatus is that is provides for a computer implemented apparatus and method of accessing and controlling a user interface for monitoring and treatment equipment in a medical environment.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

According to one embodiment of the invention, a device for monitoring physiological parameters of a medical patient includes a pneumatic system configured to be coupled to a patient to provide a regulated gas thereto, a computer coupled to the pneumatic system and configured to regulate gas to the patient via the pneumatic system, and a touchscreen monitor coupled to the computer. The touchscreen monitor includes a first graphical user interface (GUI) having a first display, and a second GUI having a second display different from the first display and configured having interaction fields to enable parameters to be input therewith. The device includes a first trigger configured to switch at least from the first GUI to the second GUI.

According to another embodiment of the invention, a method of monitoring physiological parameters of a patient includes displaying a first data display in a first graphical user interface (GUI) of a touchscreen monitor, and triggering the touchscreen monitor to display a second GUI that includes a second data display that is different from the first data display, and includes one or more interaction fields in the second GUI, wherein the one or more interaction fields are configured to set parameters of a device for monitoring the physiological parameters of the medical patient.

According to yet another embodiment of the invention, a non-transitory computer readable storage medium having stored thereon a computer program representing a set of instructions that when executed by a computer causes the computer to display a first graphical user interface (GUI) of a touchscreen display having a first data display, display a second GUI of the touchscreen display having a second data display that is different from the first data display, and having one or more interaction fields for parameter input, and receive an input from a first trigger that is configured to switch at least between the first GUI and the second GUI.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A device for monitoring physiological parameters of a medical patient, the device comprising:
a pneumatic system configured to be coupled to a patient to provide a regulated gas thereto;
a computer coupled to the pneumatic system and configured to regulate gas to the patient via the pneumatic system;
a touchscreen monitor coupled to the computer, the touchscreen monitor comprising:
a first graphical user interface (GUI) having a first display; and
a second GUI having a second display different from the first display and configured having interaction fields to enable parameters to be input therewith; and
a first trigger configured to switch at least from the first GUI to the second GUI.

2. The device of claim 1 wherein the first data display is limited to displayed data and does not include interaction fields that provide a capability for parameter input.

3. The device of claim 1 wherein the first trigger is one of a radio-frequency identification (RFID) tag and a bluetooth device.

4. The device of claim 3 comprising a second trigger configured to switch to the first GUI when no users are detected within the vicinity of the device.

5. The device of claim 4 wherein the second trigger is a screen saver configured to switch to the first GUI after a pre-set period of time.

6. The device of claim 1 wherein the first trigger is a proximity sensor.

7. The device of claim 6 wherein the proximity sensor includes one of an infrared sensor, an optical sensor, and a laser.

8. The device of claim 1 wherein the first trigger is configured to switch to the second GUI by tapping the touchscreen display.

9. The device of claim 1 wherein the second GUI includes a lock-out activated by a user, the lock-out configured to prevent triggering a change from the second GUI.

10. The device of claim 1 comprising a superuser trigger configured to supersede the first trigger and display a third GUI that is configured to enable superuser inputs.

11. The device of claim 1 wherein the device is one of a ventilator, an anesthesia machine, and a vital signs monitoring machine.

12. A method of monitoring physiological parameters of a patient, the method comprising:
displaying a first data display in a first graphical user interface (GUI) of a touchscreen monitor; and
triggering the touchscreen monitor to display a second GUI that includes a second data display that is different from the first data display, and includes one or more interaction fields in the second GUI, wherein the one or more interaction fields are configured to set parameters of a device for monitoring the physiological parameters of the medical patient.

13. The method of claim 12 wherein triggering the touchscreen monitor comprises triggering the touchscreen monitor via one of a proximity sensor, a radio-frequency identification (RFID) tag, and a bluetooth device.

14. The method of claim 13 wherein triggering the touchscreen monitor via the proximity sensor comprises triggering the display via one of an infrared sensor, an optical sensor, and a laser.

15. The method of claim 12 comprising triggering the touchscreen monitor to display the second GUI by tapping the touchscreen display.

16. The method of claim 12 comprising triggering the touchscreen display to display the first GUI after a pre-set period of time.

17. The method of claim 12 comprising locking out other users from triggering the touchscreen display to change from the second GUI.

18. The method of claim 12 comprising triggering the touchscreen display to display a third GUI by superseding other users via a superuser identifier.

19. A non-transitory computer readable storage medium having stored thereon a computer program representing a set of instructions that when executed by a computer causes the computer to:
display a first graphical user interface (GUI) of a touchscreen display having a first data display;
display a second GUI of the touchscreen display having a second data display that is different from the first data display, and having one or more interaction fields for parameter input; and
receive an input from a first trigger that is configured to switch at least between the first GUI and the second GUI.

20. The computer readable storage medium of claim 19 wherein the received input from the first trigger comprises an input from one of a proximity sensor, a radio-frequency identification (RFID) tag, and a bluetooth device.

21. The computer readable storage medium of claim 19 wherein the computer is configured to display the first GUI of the touchscreen display after a pre-set period of time.

22. The computer readable storage medium of claim 19 wherein the computer is further programmed to detect when the touchscreen display is tapped, and switch to the second GUI when the touchscreen display is tapped.

23. The computer readable storage medium of claim 19 wherein the computer is programmed to receive lock-out information from a user that, when activated, prevents other users from triggering a change in a currently displayed GUI.

24. The computer readable storage medium of claim 19 wherein the computer is programmed to receive an input from a super-user that supersedes other displays and causes the computer to display a third GUI.
